(19) United States
(12) Patent Application Publication
(10) Pub. No.: US 2009/0041815 A1
(43) Pub. Date: Feb. 12, 2009

(54) ASSEMBLY COMPRISING A SUBSTRATE COMPRISING BIOCELLULOSE, AND A POWDERED COSMETIC COMPOSITION TO BE BROUGHT INTO CONTACT WITH THE SUBSTRATE

(75) Inventor: Jean-Yves Legendre, Paris (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/165,924

(22) Filed: Jul. 1, 2008

Related U.S. Application Data
(60) Provisional application No. 60/929,737, filed on Jul. 11, 2007.

(30) Foreign Application Priority Data
Jul. 2, 2007 (FR) 07 56216

(51) Int. Cl. A61K 9/14

(52) U.S. Cl. 424/401

(57) ABSTRACT

The present invention provides an assembly comprising:
a substrate comprising biocellulose;
a powdered cosmetic composition that is at least partially hydrosoluble, to be brought into contact with the substrate.

This non provisional application claims the benefit of French Application No. 07 56216 filed on Jul. 2, 2007 and U.S. Provisional Application No. 60/929,737 filed on Jul. 11, 2007.

The present invention relates to methods of preparing a cosmetic composition and to assemblies allowing a cosmetic composition to be prepared and applied.

### BACKGROUND

There is sometimes a need to increase the contact time between an application site and a cosmetic composition in order to optimize the efficacy thereof.

To this end, various substrates have been developed with the aim of being impregnated with a composition and then applied to the site to be treated. The composition that is thus impregnated into the substrate diffuses more slowly towards the site of action compared with the composition without the substrate. Publications EP-A-1 095 589, EPA-1 338 264, U.S. Pat. No. 6,702,792, and WO-A-06/053332 disclose facial treatment masks with a nonwoven substrate impregnated with emulsion type cosmetic compositions presenting whitening or anti-ageing actions.

Similar products for body tightening applications have also recently appeared on the market (anti-cellulite wraps, Body Sculptess, Lancôme).

Other substrates such as natural polysaccharide gels may also be used to produce impregnated articles, as disclosed in publication US-2003/0113356.

While they are practical in use, such cosmetic articles may suffer from the disadvantage of containing relatively high concentrations of preservatives; the various substrates employed are prone to microbial contamination.

Further, manufacturing them requires a step of impregnating the cosmetic composition onto the substrate. That impregnation is occasionally difficult to accomplish in a uniform manner and in particular may change over time and result in sedimentation of the cosmetic formula.

The following publications: US2001/0028894, U.S. Pat. No. 6,730,317, and WO-A-03/075820 disclose other systems for prolonged administration of cosmetic substances such as patches. The active ingredients are then directly incorporated into the matrix of the patch, which is then placed on the site of action.

The choice of matrix dictates the adhesiveness of the patch. The use of chemically cross-linked polymers such as acrylic polymers may cause undesirable reactions on the skin and does not enable ecologically responsible articles to be obtained.

Further, such systems suffer from the major disadvantages of being difficult to formulate with all types of active ingredients and at high concentrations, as well as having a low diffusion rate of the active ingredients towards the site of action because the kinetics are relatively slow.

Finally, those patches are not very exciting cosmetically and no immediate benefit is produced.

The aim of the invention is to propose a novel manner of preparing and/or applying a cosmetic composition, overcoming some or all of the above disadvantages.

In one aspect, the invention provides an assembly comprising:
a substrate comprising biocellulose;
a powdered cosmetic composition that is at least partially hydrosoluble, to be brought into contact with the substrate.

The above assembly may be offered to the consumer in a single package, for example a box, blister pack, kit, sachet or receptacle with multiple compartments.

The substrate may be moist or, in a variation, it may be dry and intended to be rehydrated, in particular extemporaneously. The assembly may then include an aqueous solution for rehydration of the substrate. In a variation, the substrate is rehydrated with water.

The aqueous solution may optionally include at least one active ingredient that is incompatible with at least one active ingredient contained in the composition.

The aqueous solution may include mineral or thermal water or plankton water, inter alia. With plankton water, the powdered cosmetic composition may contain plankton, for example in lyophilized form.

The powdered cosmetic composition may have a mean grain size of 5 μm [micrometer] to 1000 μm, or even of 50 μm to 500 μm.

The powdered cosmetic composition may contain at most 10% by weight of water, or even at most 5% by weight of water, or it may even be completely anhydrous.

### SUMMARY

The invention allows the water contained in the moist biocellulose to be used to at least partially dissolve the cosmetic composition in a rapid, clean, and entertaining manner.

A powdered form with low water content, or even completely anhydrous, can facilitate preservation of the composition, even in the absence of preservatives.

The substrate may advantageously be contained in sterile packaging, especially when in the moist form. When it is dry, preservation of the substrate may be facilitated.

The powdered cosmetic composition may be contained in any suitable packaging that is suitable for single or multiple use, and that may optionally close in a sealed manner, for example a sachet or a bottle, optionally provided with measuring out and/or dispensing means, for example a sieve.

When present, the aqueous substrate rehydration solution may also be contained in any appropriate packaging that is suitable for single or multiple use, and that may optionally close in a sealed manner, for example a sachet, a bottle or an ampoule, optionally provided with measuring out and/or dispensing means.

If appropriate, the powdered composition and the aqueous solution may be contained in two compartments of the same packaging.

In another aspect, the invention provides a method of preparing a cosmetic composition, in particular extemporaneously, comprising the steps consisting in:
bringing a moist substrate comprising biocellulose into contact with a powdered cosmetic composition that is at least partially hydrosoluble, to cause said powdered cosmetic composition to dissolve at least partially in contact with the substrate.

The substrate may be dry and may be rehydrated extemporaneously. In a variation, the substrate is already moist.

The invention also provides a method of non therapeutic cosmetic treatment using a composition prepared as above.

The powdered cosmetic composition may be deposited on the substrate and then it may be applied to keratinous material. In a variation, the powdered cosmetic composition may be applied to the keratinous material and then covered with the substrate.

The substrate may be left in place on the keratinous material for a period in the range 5 minutes to 12 hours, for example.

### Substrate

The substrate comprises biocellulose.

Biocellulose is a material obtained by aerobic fermentation, in an aqueous nutrient medium, of bacteria of the genus Acetobacter (also known as Gluconacetobacter) (Z. Gromet-Elhanan, S. Hestrin, Synthesis of cellulose by Acetobacter Xylinum, J. Bacteriol 85, 284-292, 1963; US 5 962 277).

The principal species used is Acetobacter Xylinum, although others may also produce biocellulose, for example Acetobacter Pasteurianus.

The conditions for culturing this bacteria to produce biocellulose are well known, in particular from the publication Factors affecting the yield and properties of bacterial cellulose, A. Krystynowicz et al., J Indus Microbial Biotech 29, 189-195, 2002.

Biocellulose can be used in the pure form or in a form which is combined with other types of fibers, for example fibers of natural origin, for example fibers from corn (maize), hemp, linseed, cotton, jute, kenaf, raffia, ramie, toquilla, sisal, rush, alfalfa, phormium, coir, wool, silk, soya, Manilla hemp, kumazasa, persimmon, kapok, burdock, cereals, or bamboo.

The biocellulose fibers may be free or bonded together and/or bonded to other fibers.

The biocellulose may be used in sheet form obtained, for example, by compacting a culture of biocellulose fibers after washing them. The thickness of the sheet is in the range 50 μm to 4000 μm, for example.

Preferably, before adding the powdered composition, the substrate contains at least 10% by weight of biocellulose relative to the total weight of substrate.

At least at the moment of use, the biocellulose based substrate may also contain, 10% to 99% of water, preferably 40% to 90%.

The substrate may optionally be associated with a support, in particular a support which allows it to be held in place on the skin.

The substrate in the form of a dry or moist sheet may be in the form of a mask or patch adapted to cover the face, eyes, the contour of the eyes, cheeks, lips, hands, or nails, inter alia.

Alternatively, the substrate in the form of a film may be associated with a flexible support with the aim of allowing easy application of the composition to the skin, nails and hair, for example in the form of a patch, a mask, a belt for the body, or an article of clothing.

In a preferred embodiment, the biocellulose based substrate is sterilized and packaged in a single-use packaging. Sterilization may be accomplished using means known to the skilled person, such as sterilizing using moist heat or ionizing radiation. As a result, no preservative is required in this embodiment.

The substrate may be packaged in the dry state for extemporaneous rehydration with water or an aqueous solution. By way of example, the solution may be thermal or mineral water, plankton water, or a solution containing at least one active ingredient, for example an active ingredient that is incompatible with an active ingredient contained in the powdered composition, i.e. that cannot be stored in contact therewith over a prolonged period without degrading.

### Powdered Cosmetic Composition

The powdered cosmetic composition may be dry, i.e. containing less than 10% by weight of water, preferably less than 5% by weight.

The composition may be in the form of a hydrophilic free powder that is extemporaneously applied to the moist biocellulose based substrate.

In examples of the invention, the powder particles can dissolve in less than 5 minutes, preferably in less than 2 minutes, on the moist substrate. Dissolution of the powder is defined as visual perception of the absence of any residual solid particles.

The mean diameter of the particles composing the powder may, for example, be in the range 5 μm to 1000 μm, preferably in the range 50 μm to 500 μm.

The composition may contain one or more cosmetic active ingredients selected from:
active ingredients in the powder form such as ascorbic acid, sodium ascorbyl phosphate, magnesium ascorbyl phosphate, ascorbyl glucoside, hyaluronic acid, salicylic acid and its derivatives, citric acid, ellagic acid, kojic acid, ferulic acid, plant extracts in powder form, dihydroxyacetone; and
active ingredients in the dissolved form adsorbed onto solid particles.

In a preferred implementation of the invention, the composition contains an active ingredient that can be readily degraded in a liquid medium and that has optimum physico-chemical stability when in the powder form.

Preferred active ingredients that may be mentioned are vitamins, dihydroxyacetone, ferulic acid, polyphenols, plant extracts and hydrolysates, extracts from microbial strains, proteins, peptides, amino acids, purine and pyrimidine bases, nucleic acids, oligonucleotides.

Furthermore, the composition may contain mineral or organic fillers such as silicas, clays, talcs or hydrated magnesium silicates, micas or aluminosilicates, clays, kaolin or hydrated aluminum silicate, boron nitrates, mineral oxides such as zinc oxide, acrylic or methacrylic acid polymers and copolymers such as the products sold with the trade name “Polytrap” by DOW CORNING, or polyamide (Nylon®) powders.

The composition may contain hydrosoluble or hydrodispersible polymers selected from (1) protein type polymers such as wheat or soya proteins; keratin, for example keratin hydrolysates and sulfonic keratins; casein; albumin; collagen; glutelin; glucagon; gluten; zein; gelatins and derivatives thereof; (2) polymers deriving from chitin or chitosan, such as anionic, cationic, amphoteric or non ionic polymers of chitin or chitosan; (3) polysaccharide polymers especially those such as (i) cellulose polymers such as hydroxyethyl cellulose, hydroxypropyl cellulose, hydroxypropylmethyl cellulose, methyl cellulose, ethylhydroxyethyl cellulose, carboxymethyl cellulose, and quaternized derivatives of cellulose; and (ii) starches and derivatives thereof; (4) acrylic polymers or copolymers such as polyacrylates, polymethacrylates and copolymers thereof; (5) vinyl polymers such as polyvinylpyrrolidones, copolymers of methylvinylether and maleic anhydride, the copolymer of vinyl acetate and crotonic acid, copolymers of vinylpyrrolidone and vinyl acetate, copolymers of vinylpyrrolidone and caprolactam, polyvinyl alcohols; (6) polymers of natural origin, which may be modified, such as gum Arabic, guar gum, xanthan derivatives, karaya gum; alginates, carrageen, ulvans and other algal colloids; glycoaminoglycans, hyaluronic acid and derivatives thereof; shellac, sandarac gum, dammar gums, gum elemis, copal gums; deoxyribonucleic acid; mucopolysaccharides such as hyaluronic acid, chondroitin sulfate; and mixtures of said polymers.

The composition may also contain one or more salts selected from salts of calcium, magnesium, sodium, potassium and mixtures thereof, and more particularly magnesium chloride, potassium chloride, sodium chloride, calcium chloride, magnesium bromide, sodium bicarbonate, magnesium phosphate, sodium phosphate, potassium phosphate, calcium sulfate, magnesium sulfate and mixtures thereof.

The composition to be applied to the moist biocellulose based substrate may be a powder with an effervescent nature.

The composition may be obtained by using one or more means which are known to the skilled person, such as grinding, spraying, sieving, mixing, dry or moist granulation, atomization, or lyophilization.

### BRIEF DESCRIPTION OF THE DRAWING

### Presentation Modes

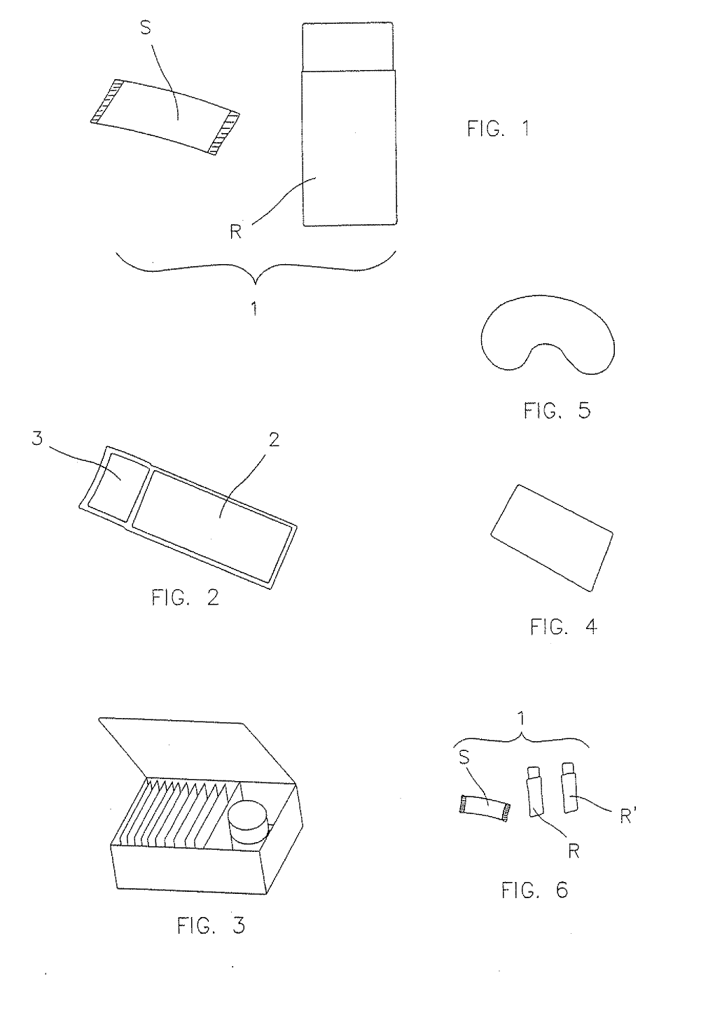

The compound may be presented in the form of a sachet or contained in a receptacle R allowing easy and homogeneous distribution over the moist substrate, while the substrate may be contained in sterile conditions in a sachet S, as shown in FIG. 1, to form an assembly 1 of the invention.

If necessary, the substrate and the composition may be packaged in two separate compartments 2, 3 of a sachet as shown in FIG. 2.

Several sachets S each comprising a single-use substrate may be offered to the consumer in a box 4 with at least one receptacle R containing the composition, as shown in FIG. 3.

The substrate may be produced in any form which is suitable for the region to be treated, for example rectangular as shown in FIG. 4 or kidney-shaped as shown in FIG. 5, for example for treating the eye contour region.

### MORE DETAILED DESCRIPTION

The assembly may be offered to the consumer with the substrate in the dry state, the powdered composition and an aqueous solution to rehydrate the substrate.

This aqueous solution is, for example, contained in a receptacle R′, as shown in FIG. 6, the receptacle R′ being proffered to the consumer in the same packaging as the substrate and the powdered composition.

### EXAMPLES

### Comparative Examples

A sheet of biocellulose was obtained by static fermentation in an aerobic medium of a strain of Acetobacter Xylinum (strain ATCC 23767) using the method described in “Factors affecting the yield and properties of bacterial cellulose”, A Krystynowicz et al., J Indus Microbiol Biotech 29, 189-195, 2002.

The biocellulose plaque obtained after fermentation was washed with distilled water, washed with a solution of dilute sodium hydroxide then neutralized with an acetic acid solution. After washing with distilled water, the biocellulose plaque was in the form of a sheet about 2 mm [millimeters] thick. The biocellulose then underwent moist heat sterilization in an autoclave at 121° C. for 20 minutes.

5 g [gram] of a powder composed of sodium alginate, calcium sulfate, potassium phosphate and silica were deposited on the surface either of a sheet of biocellulose or a hydrogel based on polyacrylic acid.

The speed at which the powder was wetted and the time after which particles of powder were no longer visible were noted.

The following results were obtained:

| | | Time after which |
|---|---|---|
| | | particles of |
| | Wetting time of | powder no longer |
| | powder | visible |
| BIOCELLULOSE | Immediate | 45 sec |
| ACRYLIC HYDROGEL | >2 min | >2 min |

Thus, it can be seen that biocellulose can dissolve the powder almost instantaneously, much faster than a hydrogel.

### Example 1

### Facial Treatment Mask for an Exfoliant and Brightening Effect

A biocellulose mask was obtained by cutting a biocellulose sheet with a punch. A powder composed of 0.05 g of salicylic acid, 0.5 g of ascorbic acid, 4 g of monohydrated citric acid and 5 g of sodium bicarbonate was distributed extemporaneously and homogeneously over the mask and the mask was immediately applied to the face for a period of 15 minutes.

### Example 2

### Purifying Hot Clay Mask

A biocellulose mask was obtained by cutting a biocellulose sheet with a punch.

A powder composed of 4 g of green clay, 0.5 g of kaolin and 2 g of anhydrous magnesium sulfate was distributed extemporaneously and homogeneously over the mask.

The mask was immediately applied to the face for a period of 15 minutes.

### Example 3

### Anti-Wrinkle Patch

A 15 cm² [square centimeter] plaque of biocellulose obtained by fermenting Acetobacter Xylinum was dried for 24 hours at 40° C. to obtain a dry sheet.

Further, a strain of Vitreoscilla Filiformis thermal plankton was cultivated at 26° C. for 48 h under the conditions described in publication WO-A-94/02158.

The biomass was separated from the culture medium by centrifuging, as described in U.S. Pat. No. 6,242,229.

The culture medium (A), also known as plankton water, was sterilized at 121° C. for 20 minutes. The biomass (B) was transformed into lyophilized powder after freezing and sublimation.

The dry biocellulose sheet was hydrated extemporaneously with 2 ml [milliliter] of sterile culture medium (A), then 0.1 g of lyophilized biomass (B) was applied to the hydrated biocellulose sheet.

The biocellulose patch containing the extemporaneously reconstituted thermal plankton was then applied to the face for an anti-wrinkle effect.

Clearly, the invention is not limited to the examples shown.

The expression “comprising a” should be understood as being synonymous with “comprising at least one” unless otherwise specified.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. An assembly comprising:
a substrate comprising biocellulose;
a powdered cosmetic composition that is at least partially hydrosoluble, to be brought into contact with the substrate.

2. An assembly according to claim 1, the substrate being moist.

3. An assembly according to claim 1, the substrate being dry and intended to be rehydrated.

4. An assembly according to claim 3, comprising an aqueous solution to rehydrate the substrate.

5. An assembly according to claim 4, the aqueous solution comprising at least one active ingredient that is incompatible with at least one active ingredient contained in the composition.

6. An assembly according to claim 4, the aqueous solution comprising mineral or thermal water, or plankton water.

7. An assembly according to claim 1, the powdered cosmetic composition having a mean grain size in the range 5 μm to 1000 μm.

8. An assembly according to claim 7, the powdered cosmetic composition having a mean grain size in the range 50 μm to 500 μm.

9. An assembly according to claim 1, the powdered cosmetic composition containing at most 10% by weight of water.

10. An assembly according to claim 9, the powdered cosmetic composition containing at most 5% by weight of water.

11. An assembly according to claim 1, the substrate being contained in a sterile packaging.

12. An assembly according to claim 1, the powdered cosmetic composition containing at least one active ingredient selected from active ingredients in powdered form such as vitamins, ascorbic acid, sodium ascorbyl phosphate, magnesium ascorbyl phosphate, ascorbyl glucoside, hyaluronic acid, salicylic acid and derivatives thereof, citric acid, ellagic acid, kojic acid, ferulic acid, plant extracts and hydrolysates in powder form, dihydroxyacetone, polyphenols, extracts of microbial strains, proteins, peptides, amino acids, purine and pyrimidine bases, nucleic acids, oligonucleotides, and mixtures thereof.

13. An assembly according to claim 1, the powdered cosmetic composition containing at least one active ingredient in dissolved form and adsorbed onto solid particles.

14. An assembly according to claim 1, the powdered cosmetic composition containing at least one mineral or organic filler.

15. An assembly according to claim 14, the filler being selected from silicas, clays, talcs or hydrated magnesium silicates, micas or aluminosilicates, clays, kaolin or hydrated aluminum silicate, boron nitrates, mineral oxides such as zinc oxide, acrylic or methacrylic acid polymers and copolymers and polyamide powders, and mixtures thereof.

16. An assembly according to claim 1, the powdered cosmetic composition comprising at least one hydrosoluble or hydrodispersible polymer.

17. An assembly according to claim 16, the polymer being selected from: protein type polymers such as wheat or soya proteins; keratin, for example keratin hydrolysates and sulfonic keratins; casein; albumin; collagen; glutelin; glucagon; gluten; zein; gelatins and derivatives thereof; polymers deriving from chitin or chitosan, such as anionic, cationic, amphoteric or non ionic polymers of chitin or chitosan; polysaccharide polymers especially those such as cellulose polymers such as hydroxyethyl cellulose, hydroxypropyl cellulose, hydroxypropylmethyl cellulose, methyl cellulose, ethylhydroxyethyl cellulose, carboxymethyl cellulose, and quaternized derivatives of cellulose; and starches and derivatives thereof; acrylic polymers or copolymers such as polyacrylates, polymethacrylates and copolymers thereof; vinyl polymers such as polyvinylpyrrolidones, copolymers of methylvinylether and maleic anhydride, the copolymer of vinyl acetate and crotonic acid, copolymers of vinylpyrrolidone and vinyl acetate, copolymers of vinylpyrrolidone and caprolactam, polyvinyl alcohols; polymers of natural origin, which may be modified, such as gum Arabic, guar gum, xanthan derivatives, karaya gum; alginates, carrageen, ulvans and other algal colloids; glycoaminoglycans, hyaluronic acid and derivatives thereof; shellac, sandarac gum, dammar gum, gum elemis, copal gums; deoxyribonucleic acid; mucopolysaccharides such as hyaluronic acid, chondroitin sulfate; and mixtures of said polymers.

18. An assembly according to claim 1, the powdered cosmetic composition comprising at least one salt.

19. An assembly according to claim 18, the salt being selected from salts of calcium, magnesium, sodium, potassium and mixtures thereof.

20. An assembly according to claim 1, the powdered cosmetic composition comprising at least one compound that can give rise to effervescence in contact with biocellulose.

21. An assembly according to claim 1, the powdered cosmetic composition being contained in a sachet.

22. An assembly according to claim 1, the powdered cosmetic composition being contained in a bottle.

23. An assembly according to claim 1, the powdered cosmetic composition being free of preservatives.

24. An assembly according to claim 1, the substrate being packaged in a sterile manner.

25. An assembly according to claim 1, the substrate comprising at least 10% by weight of biocellulose.

26. An assembly according to claim 1, the substrate being constituted by moist biocellulose.

27. An assembly according to claim 1, the substrate comprising at least 10% by weight of water.

28. An assembly according to claim 27, the substrate comprising in the range 10% to 99% by weight of water.

29. A method of preparing a cosmetic composition, comprising:
bringing a moist substrate comprising biocellulose into contact with a powdered cosmetic composition that is at least partially hydrosoluble, to cause said powdered cosmetic composition to dissolve at least partially in contact with the substrate.

30. A method according to claim 29, contact being extemporaneous.

31. A method according to claim 29, the substrate being extemporaneously moistened using water or an aqueous solution.

32. A method of cosmetic treatment using a composition prepared using the method defined in claim 29, the cosmetic composition being applied to keratinous material.

33. A method according to claim 32, the composition being initially deposited in a powdered state on the substrate before being applied to the keratinous material.

34. A method according to claim 32, the powdered cosmetic composition being initially applied to the keratinous material, then covered with the substrate.

35. A method according to claim 32, the substrate being left in place on the keratinous material for a period in the range 5 minutes to 12 hours.
